(19) United States
(12) Patent Application Publication
(10) Pub. No.: US 2021/0299392 A1
(43) Pub. Date: Sep. 30, 2021

(54) A BREATHING CIRCUIT HAVING AN EMBEDDED HEATING WIRE AND TEMPERATURE SENSOR

(71) Applicant: VINCENT MEDICAL (DONG GUAN) MANUFACTURING CO., LTD, TangXia, Dongguan, Guangdong (CN)

(72) Inventors: Jiebing XU, Tangxia, Dongguan, Guangdong (CN); Haibin YU, Tangxia, Dongguan, Guangdong (CN); Wei ZHU, Shenzhen, Guangdog (CN); Jun ZHAO, Dongguan, Guangdong (CN)

(21) Appl. No.: 17/265,616

(22) PCT Filed: Feb. 15, 2019
(86) PCT No.: PCT/CN2019/075245

§ 371 Date: Feb. 3, 2021

(51) Int. Cl. A61M 16/10; A61M 16/08

(52) U.S. Cl. CPC A61M 16/109; A61M 2205/3368; A61M 16/0875

(57) ABSTRACT

A breathing circuit contains a heating circuit (or heating wire) and a sensor, optionally a temperature sensor therein. The heating circuit (or heating wire), and/or the sensor is embedded within the conduit wall. Respiratory apparatuses containing such a breathing circuit, as well as methods of making such a breathing circuit are provided.

### FIELD OF THE INVENTION

The present invention relates to breathing circuits and more specifically to breathing circuits having embedded heating wires.

### BACKGROUND

Breathing circuits, a.k.a., breathing tubes, are well-known for use with patients who requite assistance breathing and/or who require specific mixtures of gases for breathing such as, for example, humidified air, heated air, oxygen-enriched air, etc. Breathing circuits typically are formed from a plastic gas conduit containing a lumen through which the breathing gas passes. Typically a ventilator generates an airflow which is directed into the inspiry limb of a breathing circuit which is then fed to a patient via, for example, a face mask or a nasal device for inhalation by the patient The patent then typically exhales air into the breathing circuit which passes the exhaled air into an expiry limb via a one-sway valve. The exhaled air may be passed directly- or indirectly- to the surrounding atmosphere.

When a humidifier is included in the system, then it is possible that the humid air may form condensation inside of the breathing circuit as the air typically will cool between the time it is introduced to the breathing circuit and when it is breathed in by the patient as the air will typically cool down before reaching the patient, and therefore the moisture capacity of the gas is reduced. It is therefore desirable to reduce condensation within the tube, as condensation could encourage microbial growth within the breathing circuit, could cause problems such as choking if inhaled by the patient/user, and/or cause malfunctions or short circuits of the electrical equipment.

In order to reduce condensation within the breathing circuit, a heating wire is known to be provided, either in the walls of the gas conduit and/or within the gas conduit itself. However, it is known that having wires looped within the gas conduit undesirably increases the air resistance for the gas flowing therethrough. Accordingly in some cases heating wires embedded into the wall(s) of the gas conduit are preferred.

In addition to heating wires, gas conduits also may contain one or more sensors, such said flow sensors, humidity sensors, temperature sensors, oxygen sensors, etc. to provide data to the user/hospital, etc. Such sensors typically require additional wires, holes, and/or parts integrated at one or more positions in the breathing circuit. Such sensors may also increase the complexity for the health care professionals using these systems, as the multiple worries and/or connectors must all be plugged into the correct location and connector. It has been found that such connections can increase user complexity and dis-satisfaction. In addition, the multiple connections may increase the chance of user error by the health care professional/user.

Such breathing conduit systems may be for use in a home, a hospital, an emergency care unit, etc.

Breathing circuits having sensors and/or heating wires therein are described by, for example, US Pat. App. No. 2017/0095632 A1 to Fisher & Paykel Healthcare, Ltd. of Auckland, New Zealand, published on Apr. 6, 2017; U.S. Pat. No. 9,572,949 B2 to Resmed Ltd. of Bella Vista, New South Wales, Australia published on Feb. 21, 2017; and PCT Pat. App. No WO 2017/004664 A1 to Ventific Holdings Pty. Ltd. of Chatswood, New South Wales, published on Jan. 21, 2017; U.S. Pat. No. 5,357,948 A to Heinz of Wipperfurth, Fed. Rep. Germany, published on Oct. 25, 1994.

However, the inventors herein now believe that it is desirable to reduce manufacturing complexity of such breathing circuit, while further incorporating various sensors while reducing the need for additional connectors, holes, parts, etc. Accordingly, it is also desirable to provide a breathing circuit and a method of manufacturing a breathing circuit having a heating wire which reduces the need for additional wires and connectors. Furthermore, it is desirable for a breathing circuit to have sensors at both the machine end and the patient end, while reducing the number of wires and connectors required.

### SUMMARY OF THE INVENTION

An embodiment of the present invention relates to a breathing circuit containing a gas conduit for delivering a gas, and a heater wire running substantially the length of the gas conduit. The gas conduit has a conduit wall, and the heater wire is configured to heat the gas conduit and is embedded within the conduit wall. The heater wire further contains a temperature sensor integrated therein.

An embodiment of the present invention relates to a breathing circuit containing a gas conduit for delivering a gas, a heating element, and a sensor. The gas conduit has a conduit wall surrounding a lumen. The heating circuit optionally contains a heater wire. The sensor optionally contains a temperature sensor. The sensor and the heating circuit are separated form each other. The heating circuit, the sensor, or both are embedded in the conduit wall.

An embodiment of the present invention relates to a respiratory apparatus containing the breathing circuit described herein.

An embodiment of the present invention relates to a method for forming a breathing circuit containing the steps of: forming a gas conduit containing a conduit wall, forming a heater wire, embedding the heater wire into the conduit wall, and electrically-connecting a plurality of sensors to the heater wire. The heater wire is an insulated heater wire, and the sensor optionally contains a temperature sensor.

Without intending to be limited by theory, it is believed that the embedding of a heater wire, a sensor, or both into the conduit wall provides significant advantages over, for example, where the heater wire is simply placed in the lumen of the has conduit. Specifically, it is believed that the embedded heater wire/sensor/both provides reduced air resistance as compared to where the heater wire/sensor/both are merely placed within the conduit. Furthermore, the embedding of the heater wire/sensor/both may lead to a reduced chance of corrosion, and/or failure/malfunctions of the electronics. In addition, the embedding of the heater wire/sensor/both may lead to a reduced surface area for microbes, bacteria, etc. to stick onto, therefore reducing the chance of contamination within the breathing circuit. The present invention may also be easily and quickly produced, may be cheaply produced, and may provide for a plurality of sensors, or even temperature sensors at multiple locations, such as both ends; or the machine end and the patient end, of the gas conduit. It is also believed that the present invention may reduce the number of wires required to provide a plurality of sensors as well as a heating circuit/heating wire.

### BRIEF DESCRIPTION OF THE DRAWINGS

FIG. 1 shows a schematic circuit diagram of an embodiment of a circuit useful herein;

FIG. 2 shows an exploded view of an embodiment of a breathing circuit herein;

FIGS. 3a-3i show schematic diagrams embodiments of the present invention where the heater wire is embedded into the gas conduit wall;

FIGS. 4a-4i show schematic diagrams of embodiments of the present invention where NTC wire a embedded into the gas conduit wall; and

FIGS. 5a-5i show schematic diagrams of embodiments of the present invention where both the heater wire and NTC wire are embedded into the gas conduit wall.

The figures herein are for illustrative purposes only and are not necessarily drawn to scale.

### DESCRIPTION OF THE PREFERRED EMBODIMENTS

Unless otherwise specifically provided, all measurements are made in metric units. Furthermore, all percentages, ratios, etc. herein are by weight, unless specifically indicated otherwise.

The gas useful herein typically includes air, and/or air enriched with oxygen gas, as desired. The gas may be at the ambient room temperature, at higher than room temperature, or lower that room temperature, as desired. The gas herein may be at a higher pressure, than the surrounding environment, or may be at the ambient pressure as the surrounding environment. The gas may be humidified gas, drier than the ambient humidity, or may be at the ambient humidity as desired.

As used herein, the term “heater wire” indicates the wire through which electricity flows and which increases in temperature so as to generate heat. As used herein, the term “heater circuit” includes both the heater wire itself as well as other electronics, such as the circuitry, etc.

A breathing circuit contains a gas conduit for delivering a gas, typically to a patient. The gas conduit contains a conduit wall, typically surrounding a lumen through which the gas passes. A heater wire runs substantially the length of the gas conduit so as to heat the gas within the gas conduit and thereby reduce and/or prevent condensation of moisture within the gas conduit and/or the lumen of the gas conduit. The heater wire may be embedded within the conduit wall and/or located within the lumen. In an embodiment herein, the heater wire further contains a temperature sensor integrated therein.

The present invention relates breathing circuits, and especially breathing circuits positioned between a machine and a patient, and intended to deliver a gas from the machine to the patient. The breathing circuit's gas conduit is typically a long tube having an inner surface forming a lumen for transporting the gas and an outer surface opposite the inner surface. The gas conduit may possess a first conduit end which may be attached to or close to a machine such as a ventilator, a humidifier, etc. (a.k.a., the machine end), and a second conduit end opposite the first conduit end which is nearer to the patient (a.k.a., the patient end). The conduit is typically formed of a plastic, such as a thermoplastic, a resin, a polymeric material, etc. Such plastics are known in the art and typically include for example, polyethylene terephthalate, polypropylene, polyethylene, ethyl vinyl acetate, polyolefin, polyvinyl chloride, and a combination thereof or a low-density polyethylene polymer and an ethyl vinyl acetate copolymer, a blend of a polypropylene polymer and an ethyl vinyl acetate copolymer, a blend of polyolefin elastomer polymers and a polyvinyl chloride polymer, and a combination thereof. Furthermore, the plastic may incorporate an anti-microbial compound by, for example, containing a coating, integrating the anti-microbial compound into the plastic, etc.

The lumen though which the gas passes may be corrugated, or may be smooth. In an embodiment herein, the lumen is a substantially smooth lumen, and the gas conduit is formed by extrusion.

The heater wire useful heron is embedded within the gas conduit wall and is not, for example, suspended or strung within the lumen of the gas conduit. The heater wire may be embedded within the conduit wall, and/or may be spirally-bound; embedded, and/or otherwise located into the conduit wall. Accordingly, one skilled in the art understands that if removed and stretched out, then the heater wire will typically be significantly longer than the gas conduit within which it is embedded. In an embodiment herein, the actual length of the heater wire is from about 1.5 times to about 20 times longer; or from about 2 times to about 15 times longer; or from about 2.5 times to about 10 times longer, than the length of the gas conduit within which it is embedded. It is believed that such a configuration is desirable as it reduces air resistance caused by the heating wires within the lumen. In an embodiment herein, the heater wire is embedded within a ridge on the outer surface of the gas conduit. In an embodiment herein the ridge is formed, or substantially simultaneously-formed, by co-extrusion of the ridge along with the gas conduit. In an embodiment herein, the heater wire and/or the ridge containing the heater wire is spirally-wrapped or concentrically-wrapped around the gas conduit and/or the lumen. The heater wire runs substantially the length of the gas conduit and is configured so as to heat the gas conduit; or to provide heating throughout the gas conduit.

In an embodiment herein, the gas conduit is intended for transporting oxygen-enriched air, and/or an oxygen and air mixture. Accordingly, it is highly important to avoid any sparks or other short-circuits which could contact the oxygen-enriched air and cause a fire. Accordingly, it is believe that by embedding the heater wire, the sensor wire, or both the heater wire and the sensor wire within the gas conduit wall, the chances for a spark and/or a fire are significantly decreased.

The heater wire further contains a sensor; or a temperature sensor, integrated therein; or a sensor; or a temperature sensor, in parallel to the heater wire and/or the heater circuit. In an embodiment herein, the heater wire and/or the heater circuit contains a plurality of sensors; or temperature sensors; or from about 2 to about 4 sensors; or temperature sensors; or from about 2 to about 3 sensors; or temperature sensors, or about 2 sensors; or temperature sensors. In an embodiment herein, the heater wire contains a sensor; or a temperature sensor, at the patient end and a separate sensor; or temperature sensor, at the machine end. Without intending to be limited by theory, it is believed that such a breathing circuit which contains a sensor; or a temperature sensor, at both the machine end and patient end provides significantly more information which can help determine the efficacy of the humidification, temperature distribution, etc. The sensor useful herein may include, for example, a temperature sensor, a gas speed sensor, a humidity sensor, a CO₂ sensor, a O₂ sensor, and a combination thereof; or a temperature sensor, a humidity sensor, a CO₂ sensor, a O₂ sensor, (what other types of sensors are preferred?) and a combination thereof; or a temperature sensor, a humidity sensor, (what other types of sensors are most preferred?) and a combination thereof.

It is believed that such multiple sensors may help to ensure, for example, that the temperature of the heated gas actually reaching the patient is within the desired temperature range. This in turn may reduce and/or prevent overly hot gasses from adversely-affecting, or even burning, the patient's respiratory tract. This may also reduce and/or prevent gases which are too cold or otherwise possessing the wrong characteristic(s), from being breathed in by the patient as well. It is also believed that this may further reduce energy requirements, and/or reduce condensation within the tube.

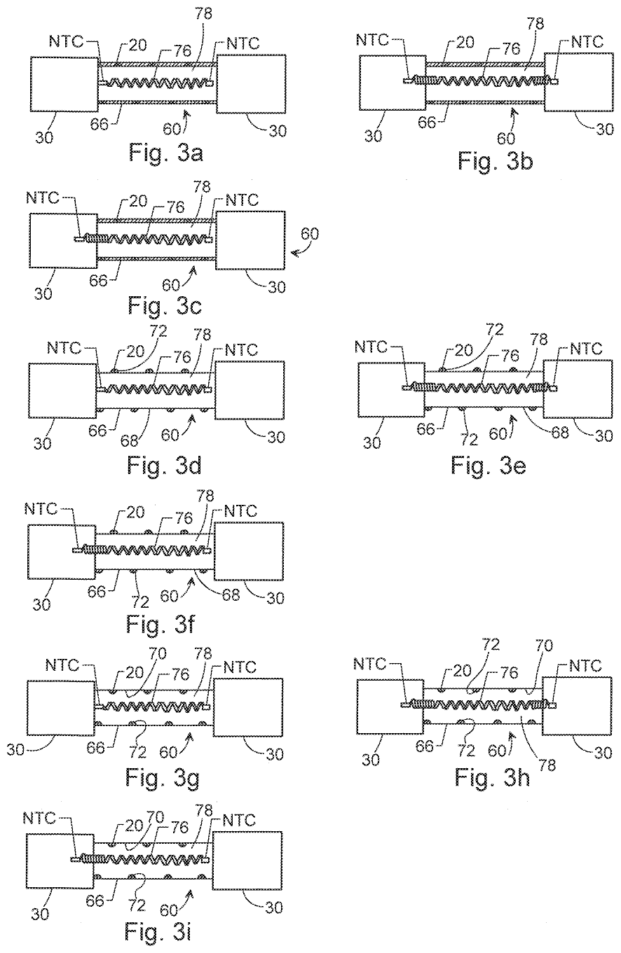

In an embodiment herein, a sensor; or a plurality of sensors; or substantially all of the sensors, are located in the gas conduit's lumen (see FIG. 3a at 78). It is also believed that when a/the sensors are within the gas conduit's lumen (see FIG. 3a at 78), then it/they are in direct contact with the gasses passing through. Therefore, they provide a much more direct and accurate measurement of the actual temperature of the gas. It is also believed that when the sensors are provided in the gas conduit's lumen (see FIG. 3a at 78), then they would be removable and likely disposed of when the gas conduit is changed. Accordingly, this reduces the chance of propagation and/or spreading of bacterial, viral, fungal, and/or other contamination.

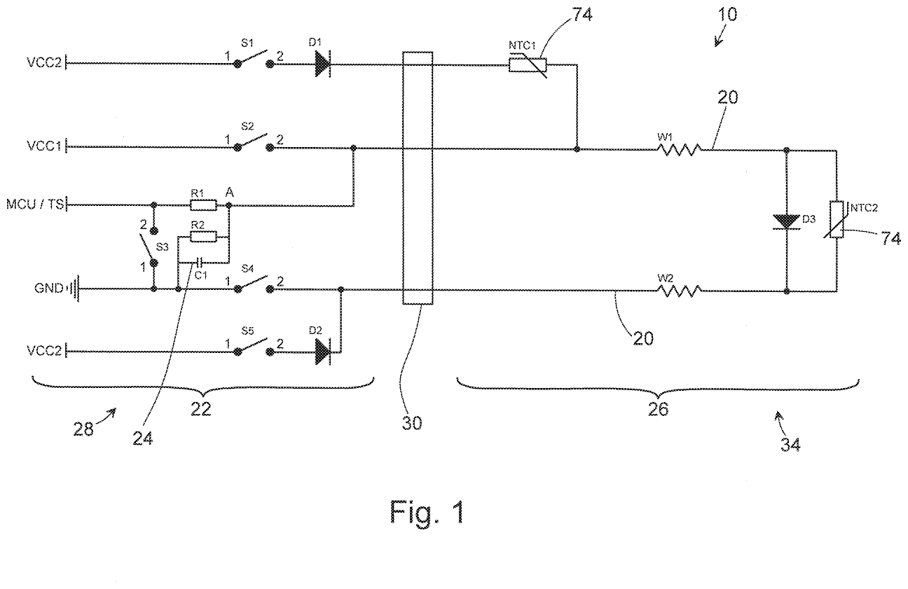

As used herein the phrase “heater wire contains (comprises) a sensor; or a temperature sensor, integrated therein” indicates that the heater wire is electrically-connected to the sensor; or the temperature sensor, by, for example, containing common circuits. For example, in FIG. 1, it can be seen that a circuit, 10, contains a heater wire, 20.

As used herein, the term “NTC” indicates a negative temperature coefficient sensor which is well-known in the art. However, other types of temperature, humidity, speed, etc. sensors are also useful herein. In addition, the temperature sensor need not necessarily be a NTC sensor, but could be, for example, a positive temperature coefficient sensor, as instead.

Firstly, FIG. 1, shows a schematic circuit diagram of an embodiment of a circuit (e.g., the electrical circuit), 10, useful herein. A sensor is a NTC sensor, 74 or NTC1 and NTC2. In order to measure the temperature of the NTC sensor, 74 or NTC1, switch, S1, is closed allowing voltage from a measurement voltage source, VCC2, to flow into the circuit and through diode, D1, flowing through resistor, R1, and completing the circuit via the temperature sensing circuitry, TS. After completing the temperature measurement of the NTC sensor, 74 or NTC1, a switch, S1, is disconnected.

Secondly in FIG. 1, to measure the temperature of the NTC sensor, 74 or NTC2, a switch, S5, is closed allowing voltage from a measurement voltage source, VCC2′, to flow into the circuit and through a diode, D2, through the NTC sensor, 74 or NTC2, to resistor R1 and then to the temperature sensing circuitry, TS. After completing the temperature measurement of the NTC sensor, 74 or NTC2, the switch, S5, is disconnected.

Thirdly in FIG. 1, the heating wire, 20, may generate heat by closing switches, S2, S3 and S4, allowing voltage from the heater voltage source, VCC1, to flow through the heater wire, 20. In order to stop the heating circuit, the switches, S2, S3 and S4, are opened thereby braking the circuit. A diode D3 provides resistance so as to allow the heating wire to heat up when the circuit is engaged. Without intending to be limited by theory, it is also believed that the diode, D3, also protects the NTC sensor, 74 or NTC2, by reducing the voltage applied to the NTC sensor, 74 or NTC2. In addition, a capacitor, 24 or Cl, is connected in parallel to resistor, R2, Thus, in FIG. 1, the NTC sensors, 74 or NTC1 and NTC2, along with the resistors, R1 and R2, and the capacitor, 24 or Cl, form the temperature measurement circuit.

More specifically, the NTC sensor, 74 or NTC2, and the resistor, R2, form a voltage division circuit. As the temperature increases, the resistance of NTC sensor, 74 or NTC2, is reduced such that the voltage at point A increases. When the temperature decreases, the resistance of NTC sensor, 74 or NTC2, increases and the voltage at point A decreases. The temperature sensing circuitry, TS, typically contains a microcontroller unit, MCU, which performs an analog-to-digital voltage conversion to obtain an analog-to-digital value which is checked in a data table to find the corresponding temperature. The resistor, R1, also serves to protect the TS/MCU port and the capacitor, 24 or Cl, provides a filtering function to stabilize the voltage at point A.

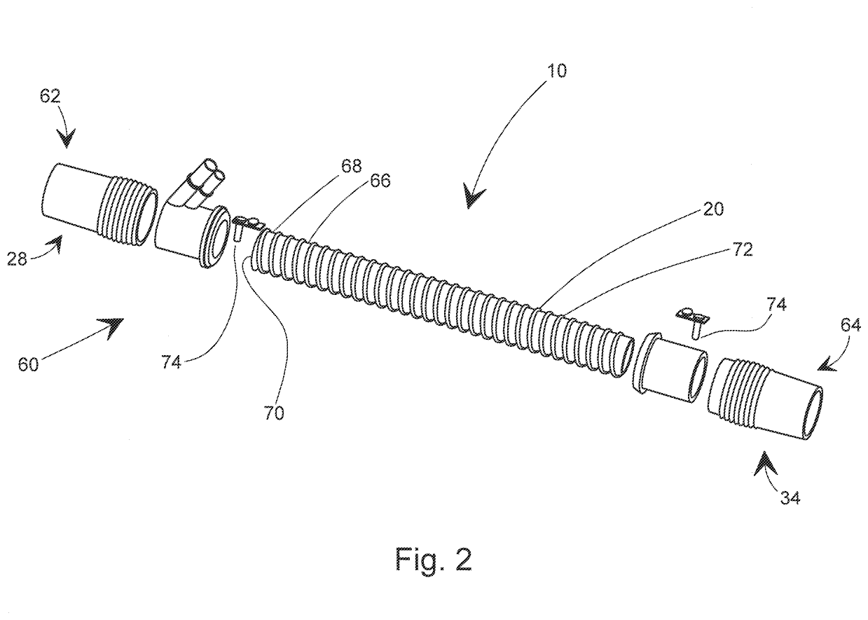

In FIG. 1, the ground is indicated by GND. Also, the host control circuit, 22, may be located at the machine end, 28. In FIG. 1, the actual heating circuit, 26, is a 3-pin heating circuit located in the gas conduit (see FIG. 2 at 60), and typically a NTC sensor, 74 or NTC2, is located at the patient end, 34. An interface, 30, isolates the components and the ends thereof and serves as a connector for the host control circuit, 22 and the heating circuit, 26.

FIG. 2 shows an exploded view of an embodiment of a gas conduit, 60, herein, it can be seen that the circuit, 10, is embedded in a gas conduit, 60. FIG. 2 shows an exploded view of a gas conduit, 60, where a first end, 62, corresponds to the machine end, 28, and a second end, 64, corresponds to the patient end, 34. It can be seen that the gas conduit contains a conduit wall, 66, containing an outer surface, 68, and an inner surface, 70, opposite the inner surface, 68. The heater wire, 20, is embedded in the outer surface, 68, within a ridge, 72. As it is embedded within the ridge, 72, the heater wire, 20, is an insulated heater wire.

Such a gas conduit, 60, is typically formed by, for example a thermoforming step. The thermoforming step may include a process selected from the group of moulding, extruding, injection, compression, and a combination thereof or moulding, extruding, and a combination thereof.

FIGS. 3a-3i show non-limiting schematic diagrams of embodiments of the present invention where the heater wire, 20, is embedded into the gas conduit wall, 66, in various manners. For example, in the cut-away view of embodiment FIG. 3a, the heater wire, 20, is located; or spirally-embedded within the conduit wall, 66, and within the width thereof—so that there are no ridges or other protrusions from the conduit are required to contain the heater wire, 20. A pair of NTC sensors, 74, are connected by an NTC wire, 76 which is strung in lumen, 78, of the gas conduit, 60. An interface, 30, is located at each end of the gas conduit, 60.

FIG. 3b shows an embodiment similar to FIG. 3a, except that the NTC sensors, 74, are located in the interfaces, 30, at either end of the gas conduit, 60. As in FIG. 3a, the heater wires are located; or embedded within the width of the conduit wall, and the NTC wire, 76, is inside of the lumen, 78, of the gas conduit, 60.

FIG. 3c shows an embodiment similar to FIG. 3a and FIG. 3b, except that only a single NTC sensor, 74, is located at the interface, 30.

FIGS. 3d-3f show cut-away views of embodiments of a gas conduit, 60, with a heater wire, 20, embedded within a ridge, 72, spirally-located on the outer surface, 68, of a conduit wall, 66. The NTC sensors, 74, are connected together by a NTC wire, 76. In FIG. 3d, the NTC sensors, 74, are located within the gas conduit's, 60, lumen, 78, while in FIG. 3e, both NTC sensors, 74, are located within the interfaces, 30. In FIG. 3f, one NTC sensor, 74, is located in the interface, 30, while the other one is located in the lumen, 78.

FIGS. 3g-3i show cut-away views of embodiments of a gas conduit, 60, with a heater wire, 20, embedded within a ridge, 72, spirally-located on the inner surface, 70, of a conduit wall, 66. The NTC sensors, 74, are connected together by a NTC wire, 76. In FIG. 3g, the NTC sensors, 74, are located within the gas conduit's, 60, lumen, 78, while in FIG. 3h, both NTC sensors, 74, are located within the interfaces, 30. In FIG. 3i, one NTC sensor, 74, is located in the interface, 30, while the other one is located in the lumen, 78.

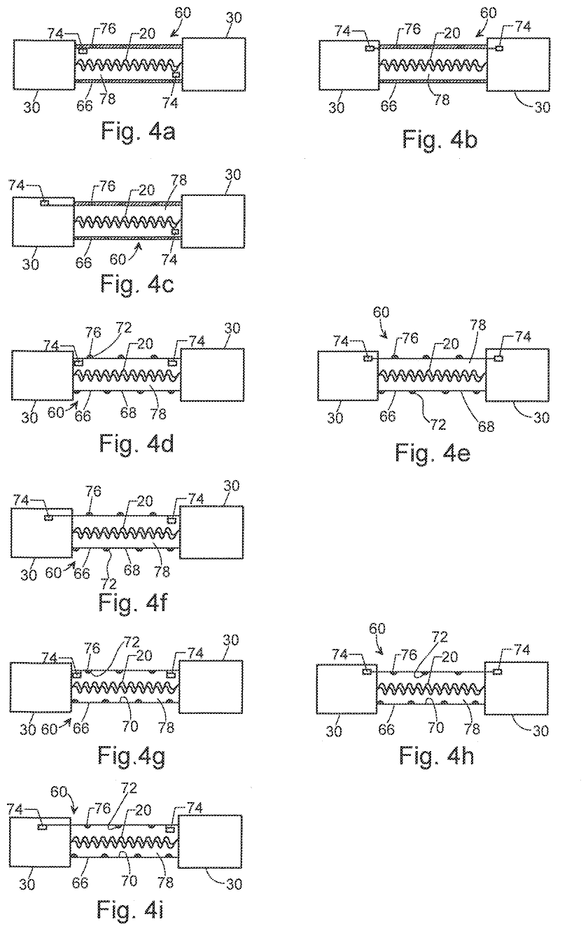

FIGS. 4a-4i show schematic diagrams of embodiments of the present invention where NTC wire is embedded into the gas conduit wall Specifically, FIGS. 4a-4c show cut-away embodiments of a gas conduit, 60, useful herein showing two NTC sensors, 74, connected by a NTC wire, 76, spirally-embedded in the conduit wall, 66, while the heater wire, 20, is located in the lumen, 78, of the gas conduit, 60. Interfaces, 30, are located at each end of the gas conduit, 60. There are no ridges or other protrusions from the conduit required to contain the NTC wire, 76. FIG. 4a shows an embodiment where the NTC sensors, 74, are located within the lumen, 78.

FIG. 4b shows an embodiment where both NTC sensors, 74, are located; or spirally-embedded, in the interfaces, 30 at each end of the gas conduit, 60.

FIG. 4c shows an embodiment where one NTC sensor, 74, is located; or spirally-embedded, in an interface, 30 at one end of the gas conduit, 60, while the other NTC sensor, 74, is located within the lumen, 78.

FIGS. 4d-4f show cut-away embodiments of a gas conduit, 60, useful herein showing two NTC sensors, 74 connected by a NTC wire, 76, embedded in a ridge, 72, spirally-located on the outer surface, 68, of the conduit wall, 66. The heater wire, 20, is located in the lumen, 78, of the gas conduit, 60. Interfaces, 30, are located at each end of the gas conduit, 60. FIG. 4d shows an embodiment where the NTC sensors, 74, are located within the lumen, 78.

FIG. 4e shows an embodiment where both NTC sensors, 74, are located; or embedded, in the interfaces, 30 at each end of the gas conduit, 60.

FIG. 4f shows an embodiment where one NTC sensor, 74, is located; or embedded, in an interface, 30 at one end of the gas conduit, 60, while the other NTC sensor, 74, is located within the lumen, 78.

FIGS. 4g-4i show cut-away embodiments of a gas conduit, 60, useful herein showing two NTC sensors, 74 connected by a NTC wire, 76, embedded in a ridge, 72, spirally-located on the inner surface, 70, of the conduit wall, 66. The heater wire, 20, is located in the lumen, 78, of the gas conduit, 60. Interfaces, 30, are located at each end of the gas conduit, 60. FIG. 4g shows an embodiment where the NTC sensors, 74, are located within the lumen, 78.

FIG. 4h shows an embodiment where both NTC sensors, 74, are located; or embedded, in the interfaces, 30 at each end of the gas conduit, 60.

FIG. 4i shows an embodiment where one NTC sensor, 74, is located; or embedded, in an interface, 30 at one end of the gas conduit, 60, while the other NTC sensor, 74, is located within the lumen, 78.

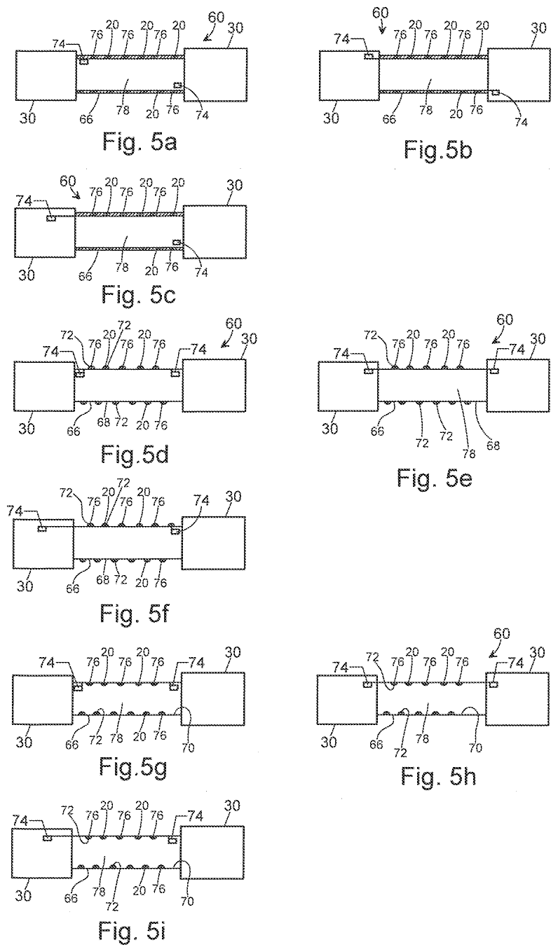

FIGS. 5a-5i show schematic diagrams of embodiments of the present invention where both the heater wire and NTC wire are embedded into the gas conduit wall. Specifically, FIGS. 5a-5c show cut-away embodiments of a gas conduit, 60, useful herein showing two NTC sensors, 74, connected by a NTC wire, 76. The NTC wire, 76, is spirally-embedded within the width of the conduit wall, 66. The heater wire, 20, is also spirally-embedded within the width of the conduit wall, 66, but offset from the NTC wire, 76. Accordingly, they form two concentric, but non-overlapping spirals along the length of the gas conduit, 60 and around the lumen, 78. The NTC 2ire, 76, connects the NTC sensors, 74. In FIG. 5a, the NTC sensors are in the lumen, 78, while in FIG. 5b, the NTC sensors are both located and/or embedded in the interfaces, 30. In FIG. 5c, a single NTC sensor, 74, is located in the lumen, 78, while the other one is located and/or embedded in the interface, 30.

FIGS. 5g-5i show cut-away embodiments of a gas conduit, 60, useful herein showing two NTC sensors, 74, connected by a NTC wire, 76. The NTC wire, 76, is spirally-embedded within a ridge, 72, which is spirally-located on the inner surface, 70, of the conduit wall, 66. The heater wire, 20, is also spirally-located on the inner surface, 70, of the conduit wall, 66, but offset from the NTC wire, 76. Accordingly, they form two concentric, but non-overlapping spirals along the length of the gas conduit, 60 and around the lumen, 78. The NTC wire, 76, connects the NTC sensors, 74. In FIG. 5d, the NTC sensors are in the lumen, 78, while in FIG. 5e, the NTC sensors are both located and/or embedded in the interfaces, 30. In FIG. 5f, a single NTC sensor, 74, is located in the lumen, 78, while the other one is located and/or embedded in the interface, 30.

One skilled in the art understands that additional arrangements and positions of the heater circuit and/or heater wire and/or the sensors; or the NTC sensor; or the NTC wire, herein are also possible, such as, for example, the heater wire and/or heater circuit is in a ridge on the inner surface while the NTC wire is in a ridge on the outer surface, etc.

In an embodiment herein, the sensor is contained within a sensor circuit, which may further include, for example, control feeds, wires, circuitry, etc. related to the sensor and/or its operation, data processing, data transmission, power, etc.

The method for forming a breathing circuit may include the steps of forming a gas conduit having a conduit wall, forming a heater wire wherein the heater wire is an insulated heater wire, embedding the heater wire into the conduit wall, and electrically-connecting a plurality of temperature sensors to the heater wire. In an embodiment herein, the embedding step is substantially simultaneous with the forming of the gas conduit an the forming of the heater wire. For example, the heater wire may be extruded into a plastic insulator while at the same time the gas conduit is extruded. These may then be combined simultaneously, or nearly-simultaneously as the insulated heater wire is wrapped; or spirally-wrapped, around the gas conduit. The extruded gas conduit, and the insulated heater wire are combined so quickly after extrusion that they bond together as their plastics intermingle.

In an embodiment herein, the respiratory circuit herein is contained within a respiratory apparatus selected from a respirator, a humidifier, a nebulizer, and a combination thereof; or a respirator, a humidifier, and a combination thereof; or a respirator; or a humidifier.

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided separately, or in any suitable subcombination.

1. A breathing circuit comprising:
A. a gas conduit for delivering a gas, the gas conduit comprising a conduit wall; and
B. a heater wire running substantially the length of the gas conduit, the heater wire configured to heat the gas conduit, wherein the heater wire is embedded within the conduit wall, the heater wire further comprising a temperature sensor integrated therein.

2. The breathing circuit according to claim 1, wherein the heating wire comprises a plurality of temperature sensors therein.

3. The breathing circuit according to claim 1, wherein the temperature sensor comprises a thermistor circuit.

4. The breathing circuit according to claim 1, wherein the gas conduit comprises a first conduit end and a second conduit end opposite the first conduit end, wherein the heater wire at the first conduit end comprises a thermistor circuit, and wherein the heater wire at the second conduit end comprises a thermistor circuit.

5. The breathing circuit according to claim 4, wherein the first conduit end is the machine end and wherein the second conduit end is the patient end.

6. The breathing circuit according to claim 1, wherein the heater wire is electrically-connected to the temperature sensor.

7. The breathing circuit according to claim 1 wherein the conduit wall comprises an inner surface and an outer surface, and wherein the heater wire is embedded in the outer surface.

8. A breathing circuit comprising:
A. a gas conduit for delivering a gas, the gas conduit comprising a conduit wall surrounding a lumen;
B. a heating circuit wherein the heating circuit optionally comprises a heater wire; and
C. a sensor circuit, wherein the sensor circuit optionally comprises a sensor,
wherein the sensor circuit and the heating circuit are separated from each other, wherein the heating circuit, the sensor circuit, or both are embedded in the conduit wall.

9. The breathing circuit according to claim 8, wherein the sensor circuit comprises a plurality of temperature sensors therein.

10. The breathing circuit according to claim 8, wherein the gas conduit comprises a first conduit end and a second conduit end opposite the first conduit end, wherein the plurality of temperature sensors comprises a first temperature sensor and a second temperature sensor, the first temperature sensor is located within the first conduit end of the gas conduit, and wherein the second temperature sensor is located within the gas conduit lumen and close to the second conduit end of the gas conduit.

11. A respiratory apparatus comprising the breathing circuit according to claim 1, wherein the respiratory apparatus is selected from the group consisting of a respirator, a humidifier, a nebulizer, and a combination thereof.

12. A method for forming a breathing circuit comprising the steps of:
A. forming a gas conduit comprising a conduit wall;
B. forming a heater wire, wherein the heater wire is an insulated heater wire;
C. embedding the heater wire into the conduit wall; and
D. electrically-connecting a plurality of sensors, optionally the sensors comprise a temperature sensor, to the heater wire.

13. The method for forming a breathing circuit according to claim 12, wherein the embedding step is substantially simultaneous with the forming of the gas conduit and the forming of the heater wire.

14. The method according to claim 12, wherein the forming of the gas conduit is by extrusion.
